(19) United States
(12) Patent Application Publication
(10) Pub. No.: US 2023/0293353 A1
(43) Pub. Date: Sep. 21, 2023

(54) WOUND PATCH

(71) Applicant: TAICEND TECHNOLOGY CO., LTD., Kaohsiung City (TW)

(72) Inventors: Gou-Don CHU, Tainan City (TW); Jun-Ping SHIAU, Kaohsiung City (TW); Fu-Chuan CHANG, Tainan City (TW)

(21) Appl. No.: 17/889,148

(22) Filed: Aug. 16, 2022

(30) Foreign Application Priority Data
Mar. 17, 2022 (TW) 111109833

(51) Int. Cl. A61F 13/02; A61F 13/00

(52) U.S. Cl. CPC A61F 13/0256; A61F 13/00029; A61F 13/0216

(57) ABSTRACT

A wound patch includes a main body made of a non-woven fabric and having opposite first and second surfaces, an adhesive layer disposed on one of the first and second surfaces of the main body, a waterproof layer disposed on the other one of the first and second surfaces of the main body, and a plurality of pore-forming sections penetrating through the main body, the adhesive layer, and the waterproof layer. The adhesive layer is attached to a wound when the adhesive layer and the main body are stretched by an external force.

### CROSS-REFERENCE TO RELATED APPLICATION

This application claims priority of Taiwanese Invention Patent Application No. 111109833, filed on Mar. 17, 2022.

### FIELD

The present disclosure relates to a patch, and more particularly to a wound patch.

### BACKGROUND

A conventional wound care treatment includes directly attaching a patch to a wound, which only requires removing the release paper that protects the adhesive layer of the patch, followed by swiftly attaching the patch to the wound. Use of the patch for wound care treatment is convenient and quick compared to the tedious steps of sterilization, dressing, and bandaging.

However, in the aforesaid wound care treatment of directly attaching the patch on the wound, with the exception of the patch being directly formed with a medicament layer or applying medicament to the wound before attaching the patch, the patch at best, can only isolate the wound from external environment to reduce the possibility of external contact and infection, and is not particularly helpful for wound healing.

### SUMMARY

Therefore, an object of the present disclosure is to provide a wound patch that can alleviate at least one of the drawbacks of the prior art.

According to the present disclosure, the wound patch includes a main body made of a non-woven fabric and having opposite first and second surfaces, an adhesive layer disposed on one of the first and second surfaces of the main body, a waterproof layer disposed on the other one of the first and second surfaces of the main body, and a plurality of pore-forming sections penetrating through the main body, the adhesive layer, and the waterproof layer. The adhesive layer is attached to a wound when the adhesive layer and the main body are stretched by an external force.

### BRIEF DESCRIPTION OF THE DRAWINGS

Other features and advantages of the present disclosure will become apparent in the following detailed description of the embodiment(s) with reference to the accompanying drawings, of which:

FIG. 1 is a schematic view illustrating an embodiment of a wound patch according to the present disclosure;

FIG. 2 is a partially enlarged cross-sectional view of FIG. 1, illustrating pore-forming sections of the embodiment of the wound patch;

FIG. 3 shows the elastic restoring performance of a main body of the embodiment of the wound patch;

FIG. 4 is a schematic view illustrating the embodiment of the wound patch in a state of use; and

FIG. 5 is a schematic view illustrating the embodiment of the wound in another state of use.

### DETAILED DESCRIPTION

Before the present disclosure is described in greater detail, it should be noted that where considered appropriate, reference numerals or terminal portions of reference numerals have been repeated among the figures to indicate corresponding or analogous elements, which may optionally have similar characteristics.

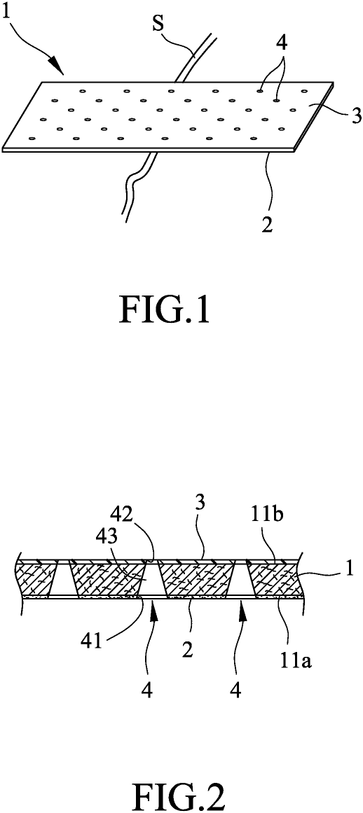

Referring to FIGS. 1 and 2, an embodiment of a wound patch configured for attachment to a wound S according to present disclosure includes a main body 1 having opposite first and second surfaces 11a, 11b, an adhesive layer 2 disposed on one of the first and second surfaces 11a, 11b of the main body 1, a waterproof layer 3 disposed on the other one of the first and second surfaces 11a, 11b of the main body 1, and a plurality of pore-forming sections 4 penetrating through the main body 1, the adhesive layer 2, and the waterproof layer 3.

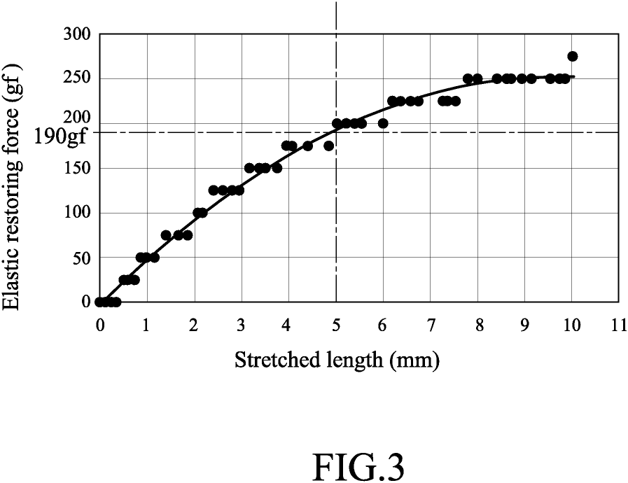

The main body 1 is made of a soft non-woven fabric. Referring to FIG. 3, the main body 1 has an elasticity coefficient ranging from 0.20 kgf/cm to 0.45 kgf/cm. Therefore, after the wound patch is attached to the wound S, the main body 1, through stretching, can still adapt to the movements and stretching of the skin, and generates an elastic restoring force to provide a closure force such that the wound S can be closed inwardly from two opposite sides thereof. As shown in FIG. 3, when in use, the main body 1 generally will not be stretched greater than 0.5 cm, which is an upper limit of a stretched length thereof. When the main body 1 was stretched 0.5 cm, the calculated elasticity coefficient is 0.38 kgf/cm, indicating that 0.38 kgf of the elastic restoring force is generated for each 1 cm of the stretched length. Therefore, when the main body 1 was stretched 0.5 cm, 190 gf of the elastic restoring force is generated. If the elastic restoring force is greater than 190 gf, the skin may be injured due to formation of reverse elastic force. In addition, when the main body 1 is attached to the wound S, in order to adapt to the rugged surface of the skin so as to achieve an optimal attachment thereto, the main body 1 has an elongation ranging from 150% to 200%. That is, if the elongation is too low, the main body 1 will not be able to adapt to the movements of the skin, and if the elongation is too high, the closure force generated from stretching of the main body 1 will be adversely affected.

The adhesive layer 2 is attached to the wound S when an external force stretches the adhesive layer 2 and the main body 1. The adhesive layer 2 has an adhesiveness ranging from 1000 g/25 mm to 2000 g/25 mm. In certain embodiments, the adhesiveness of the adhesive layer 2 is 1500 g/25 mm, such that the adhesiveness of the adhesive layer 2 is not excessively high so as to avoid damage to the outermost layer of the skin (i.e., stratum corneum) during removal of the wound patch therefrom, or such that the adhesiveness of the adhesive layer 2 is sufficient so as to ensure stable attachment to the skin. Since the elongation (i.e., stretched length) of the main body 1 may represent the softness thereof, when the main body 1 is appropriately stretched, the adhesive layer 2 may have a relatively low adhesiveness, so that the adhesive layer 2 can be stably attached to a user’s skin together with the main body 1 and can be gently removed without causing pain to the user.

The waterproof layer 3 is made of a material selected from the group consisting of polyethylene, polypropylene, polyurethane, polyacrylamide, and combinations thereof. The waterproof layer 3 prevents the main body 1 from absorbing moisture from the external environment, and thus avoids an adverse effect on the elasticity coefficient and elongation of the main body 1, and also decreases the risk of infections caused by microorganisms present in the external environment.

Each of the pore-forming sections 4 has a first end portion 41 adjacent to the adhesive layer 2, a second end portion 42 adjacent to the waterproof layer 3 and having a diameter smaller than the diameter of the first end portion 41, and a hollow portion 43 connecting the first end portion 41 and the second end portion 42. The diameter of the hollow portion 43 gradually changes along a direction from the first end portion 41 to the second end portion 42. The pore-forming sections 4 are formed with a density ranging from 10 per square cm to 20 per square cm. The diameter of the first end portion 41 ranges from 0.35 mm to 0.45 mm, and the diameter of the second end portion 42 ranges from 0.25 mm to less than 0.35 mm. A difference between the diameter of the first end portion 41 and the diameter of the second end portion 42 ranges from 0.05 mm to 0.10 mm. For example, if the diameter of first end portion 41 is 0.40 mm, the diameter of the second end portion 42 is 0.30 mm. With each of the pore-forming sections 4 having appropriate pore parameters, e.g., the diameter of the first end portion 41 differs from that of the second end portion 42, not only fluids from tissues can be discharged by capillary action to the outside, but also water droplets from the external environment will not permeate easily into the wound patch due to a low surface tension.

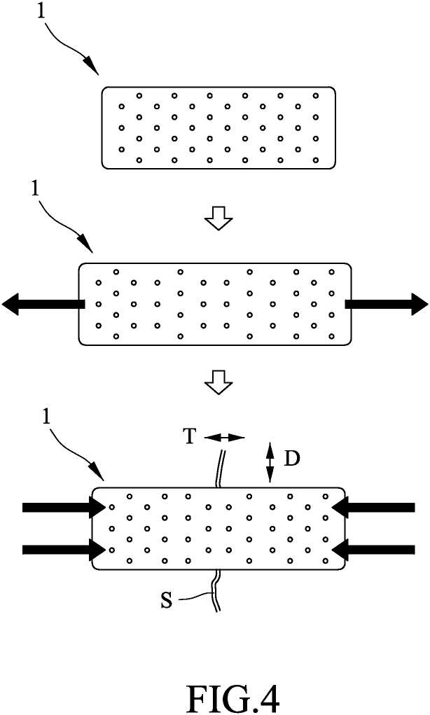

Referring to FIG. 4 in combination with FIG. 2, when the wound patch of present disclosure is in use, if the wound S extends along a first direction D and is burst open in a second direction T that is perpendicular to the first direction D, the main body 1, the adhesive layer 2 and the waterproof layer 3 are stretched, and then the wound patch is attached to the wound S along a direction parallel to the second direction T. When the wound patch of the present disclosure is attached to the wound S on a human body, the configuration of adhesive layer 2 cooperates with that of the main body 1 to provide an anchoring force so as to stably attach the wound patch to the wound S. Then, the main body 1, through stretching, generates an elastic restoring force to provide a closure force which closes the wound S, thereby forming an auxiliary force similar to suturing the wound S inwardly from the two opposite sides thereof. In addition, the pore-forming sections 4 facilitate discharge of fluids from tissue in the wound S to the outside, and cooperate with the waterproof layer 3 to avoid infiltration of sources of infections from the external environment, so that the purpose of assisting healing of the wound S can be achieved.

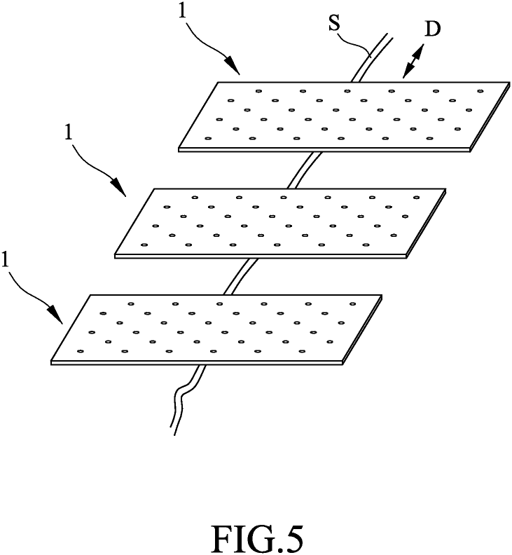

Referring to FIG. 5, it should be noted that, if the wound S has a relatively greater length along the first direction D, a plurality of the wound patch of the present disclosure may be attached spaced apart from each other at appropriate distance along the first direction D, thereby meeting the treatment requirements.

In summary, since the configuration of the adhesive layer 2 cooperates with that of the main body 1, the anchoring force thus generated allows the wound patch to be stably attached to the wound S on the human body, and since the main body 1, the adhesive layer 2 and the waterproof layer 3 are simultaneously stretched before the wound patch is attached to the wound S, the elastic restoring force generated by the main body 1 provides a closure force that closes the wound S, thereby facilitating wound healing.

In the description above, for the purposes of explanation, numerous specific details have been set forth in order to provide a thorough understanding of the embodiments. It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the disclosure has been described in connection with what is considered the exemplary embodiment, it is understood that this disclosure is not limited to the disclosed embodiment but is intended to cover various arrangements included within the spirit and scope of the broadest interpretation so as to encompass all such modifications and equivalent arrangements.

What is claimed is:

1. A wound patch, comprising:
a main body made of a non-woven fabric and having opposite first and second surfaces;
an adhesive layer disposed on one of said first and second surfaces of said main body;
a waterproof layer disposed on the other one of said first and second surfaces of said main body; and
a plurality of pore-forming sections penetrating through said main body, said adhesive layer, and said waterproof layer,
wherein said adhesive layer is attached to a wound when said adhesive layer and said main body are stretched by an external force.

2. The wound patch as claimed in claim 1, wherein each of said pore-forming sections has a first end portion adjacent to said adhesive layer, a second end portion adjacent to said waterproof layer and having a diameter smaller than the diameter of said first end portion, and a hollow portion connecting said first end portion and said second end portion, the diameter of said hollow portion gradually changing along a direction from said first end portion to said second end portion.

3. The wound patch as claimed in claim 2, wherein for each of the pore-forming sections, the diameter of said first end portion ranges from 0.35 mm to 0.45 mm, and the diameter of said second end portion ranges from 0.25 mm to less than 0.35 mm.

4. The wound patch as claimed in claim 3, wherein for each of the pore-forming sections, a difference between the diameter of said first end portion and the diameter of said second end portion ranges from 0.05 mm to 0.10 mm.

5. The wound patch as claimed in claim 1, wherein said pore-forming sections are formed with a density ranging from 10 per square cm to 20 per square cm.

6. The wound patch as claimed in claim 1, wherein an elasticity coefficient of said main body ranges from 0.20 kgf/cm to 0.45 kgf/cm.

7. The wound patch as claimed in claim 1, wherein said main body has an elongation ranging from 150% to 200%.

8. The wound patch as claimed in claim 1, wherein said adhesive layer has an adhesiveness ranging from 1000 g/25 mm to 2000 g/25 mm.
